(19) United States
(12) Patent Application Publication
(10) Pub. No.: US 2007/0016156 A1
(43) Pub. Date: Jan. 18, 2007

(54) Absorbent tampon comprising a visually distinct withdrawal member

(75) Inventors: Brian Kenneth Burgdorf, Norwood, OH (US); Michael Devon Hayes, West Chester, OH (US); Robert Paul Cassoni, Washington Twp, OH (US); Debra Lynn Hartsell, Cincinnati, OH (US)

Correspondence Address: THE PROCTER & GAMBLE COMPANY;INTELLECTUAL PROPERTY DIVISION, WINTON HILL BUSINESS CENTER - BOX 161, 6110 CENTER HILL AVENUE, CINCINNATI, OH, 45224, US

(73) Assignee: The Procter & Gamble Company

(21) Appl. No.: 11/182,303

(22) Filed: Jul. 15, 2005

(51) Int. Cl. A61F 13/20

(52) U.S. Cl. 604/385.18; 604/361

(57) ABSTRACT

A tampon that includes a primary absorbent member, secondary absorbent member and a withdrawal member that is visually distinct from the secondary absorbent member. The visual distinctiveness of the withdrawal member with respect to the secondary absorbent member is due to a difference in color, shade, pattern, reflectance, opacity, translucence, opalescence, fluorescence, luminescence, phosphorescence, chemiluminescence, whiteness and the mixtures thereof.

### FIELD OF THE INVENTION

This invention relates to absorbent tampons that include withdrawal member that is visually distinct from the secondary absorbent member.

### BACKGROUND OF THE INVENTION

A wide variety of absorbent catamenial tampons have long been known in the art. While it has been found that these tampons perform their intended function tolerably well, even the best of them do not always re-expand sufficiently, or fast enough, to provide good coverage against leakage or “bypass” failure that occurs when the menses travels along the length of the vagina without contacting the tampon. To overcome these issues, a secondary absorbent member has been used to prevent leakage and absorb bypassed fluid. However, it has been noted that consumers have been confused about the purpose of the secondary absorbent member on the tampon. Consumers have been using the secondary absorbent member as a means to withdraw the tampon. This misperception has lead to an unpleasant and unsanitary withdrawal experiences for the consumer. The visually distinct withdrawal member provides a visual cue for the consumer to perceive the secondary absorbent member as an absorbent structure and the visual distinct withdrawal member as nonabsorbent. This allows the consumer to correctly choose the visually distinct withdrawal member during the withdrawal of the tampon and allow for a more pleasant experience.

### SUMMARY OF THE INVENTION

The present invention is directed to a tampon that comprises a primary absorbent member, secondary absorbent member and a withdrawal member that is visually distinct from the secondary absorbent member. The visual distinctiveness of the withdrawal member with respect to the secondary absorbent member is due to a difference in color, shade, pattern, reflectance, opacity, translucence, opalescence, fluorescence, luminescence, phosphorescence, chemiluminescence, whiteness and the mixtures thereof.

### BRIEF DESCRIPTION OF THE DRAWINGS

While the specification concludes with claims particularly pointing out and distinctly claiming the subject matter of the present invention, it is believed that the invention will be better understood from the description taken in conjunction with the accompanying drawings, in which:

FIG. 1 is a front view of a tampon of the present invention incorporating a primary absorbent member, a secondary absorbent member and a visually distinct withdrawal member.

FIG. 2 is a perspective view a typical tampon pledget prior to compression into the primary absorbent member of a tampon of the present invention.

FIG. 3 is a perspective view of another tampon pledget prior to compression into the primary absorbent member of a tampon of the present invention in which the pledget is chevron shaped structure.

FIG. 4 is front view of the tampon of the present invention in which there is a gap between the withdrawal end of primary absorbent member and the secondary absorbent member.

FIG. 5 is a front view of the tampon of the present invention in which the secondary absorbent member is provided in the form of a plurality of discrete pieces of absorbent material.

FIG. 6 is a front view of the tampon of the present invention in which the secondary absorbent member is attached to the primary absorbent member.

FIG. 7 is a front view of the tampon of the present invention in which a portion of the secondary absorbent member is attached to the primary absorbent member and a portion of the secondary absorbent member is attached to the visually distinct withdrawal member.

FIG. 8 shows one method of making one or more tampons of the present invention in which multiple pads may be cut from a continuous strip of absorbent material.

FIG. 9 shows a pad which has been cut from the continuous strip of absorbent material of FIG. 8 and to which a visually distinct withdrawal member has been attached prior to compression.

### DETAILED DESCRIPTION OF THE INVENTION

As used herein “applicator” refers to a device or implement that facilitates the insertion of a tampon, medicament, treatment device, visualization aid, or other into an external orifice of a mammal, such as the vagina, rectum, ear canal, nasal canal, or throat. Non-limiting specific examples of such include any known hygienically designed applicator that is capable of receiving a tampon may be used for insertion of a tampon, including the so-called telescoping, tube and plunger, and the compact applicators, an applicator for providing medicament to an area for prophylaxis or treatment of disease, a spectroscope containing a microcamera in the tip connected via fiber optics, a speculum of any design, a tongue depressor, a tube for examining the ear canal, a narrow hollow pipe for guiding surgical instruments, and the like.

The term “digital tampon” refers to a tampon which is intended to be inserted into the vaginal canal with the user's finger and without the aid of an applicator. Thus, digital tampons are visible to the consumer prior to use rather than being housed in an applicator.

The term “joined” or “attached,” as used herein, encompasses configurations in which a first element is directly secured to a second element by affixing the first element directly to the second element; configurations in which the first element is indirectly secured to the second element by affixing the first element to intermediate member(s) which in turn are affixed to the second element; and configurations in which the first element is integral with the second element; i.e., the first element is essentially part of the second element.

The “outer surface” of a tampon refers to the visible surface of the (compressed and/or shaped) tampon prior to use and/or expansion. The outer surface may optionally be aesthetically textured, such as with ribs, spiraling ribs, a mesh pattern, etc. Tampons can be constructed from an absorbent material, which has been compressed and/or shaped in any or all of the width direction, the radial direction, and the axial direction, in order to provide a tampon which is of a size and stability to allow insertion within the vagina or other body cavity.

As used herein the terms “pledget” or “tampon pledget” are intended to be interchangeable and refer to a construction of absorbent material prior to the compression and/or shaping of such construction into a tampon as described above. Tampon pledgets are sometimes referred to as a tampon blank, or a softwind, and the term “pledget” is intended to include such terms as well. In general in this specification, the term “tampon” is used to refer to a finished tampon after the compression and/or shaping process. It will be recognized by those of skill in the art that in some contexts these terms are interchangeable. The different stages of tampon manufacture are described herein with an eye toward providing the greatest possible clarity. Therefore, the terms used are to assist the reader in best understanding the features of the invention and not to introduce limitations in the terms not consistent with the context in which they are used in this specification.

A tampon has a “self-sustaining shape” when a tampon pledget has been compressed and/or shaped such that it assumes a general shape and size, which is vaginally insertable, absent external forces. It will be understood by one of skill in the art that this self-sustaining shape need not, and preferably does not persist during actual use of the tampon. That is, once the tampon is inserted and begins to acquire fluid, the tampon may begin to expand and may lose its self-sustaining form.

As used herein the term “tampon,” refers to any type of absorbent structure that is inserted into the vaginal canal or other body cavities for the absorption of fluid and/or gas therefrom, to aid in wound healing, or for the delivery of active materials, such as medicaments, or moisture. The tampon may be compressed into a generally cylindrical configuration in the radial direction, axially along the longitudinal axis or in both the radial and axial directions. While the tampon may be compressed into a substantially cylindrical configuration, other shapes are possible. These may include shapes having a cross section that may be described as rectangular, triangular, trapezoidal, semi-circular, hourglass, serpentine, or other suitable shapes. Tampons have an insertion end, withdrawal end, a length, a width, a longitudinal axis and a radial axis. The tampon's length can be measured from the insertion end to the withdrawal end along the longitudinal axis. A typical compressed tampon for human use is 30-60 mm in length. A tampon may be straight or non-linear in shape, such as curved along the longitudinal axis. A typical compressed tampon is 8-20 mm wide. The width of a tampon, unless otherwise stated in the specification, corresponds to the length across the largest cylindrical cross-section, along the length of the tampon.

The term “transparent” refers to the material capability of transmitting light so that the component, or a portion thereof, can be seen as clearly as if there were no intervening material between the component and the perceiver. Furthermore, the “transparent” clarity indicates the degree of distortion of an object that can be seen through a film. (See The Wiley” Encyclopedia of packaging technology). The material is transparent or “substantially transparent” when light readily passes through. The material can be “substantially transparent” when the window is frosted or not frosted in a matte or gloss finish. When the material is frosted or not frosted in a matte or gloss finish, the frost or color can be any colors such as selected from the group consisting of blue, yellow, green, pink, white, orange, gold, rust, gray, brown, violet, and mixtures thereof.

As used herein, the term “visually distinct” refers to a difference between two points on a tampon which 8 out of 10 people, having average 20/20 vision and who are not color blind, can perceive at a distance of less than 12 inches or 30.5 cm. Visually distinct can include but is not limited to a difference in color, shade, pattern, texture, reflectance, opacity, translucency, opalescence, fluorescence, luminescence, phosphorescence, chemiluminescence, whiteness and the like. The visual distinctness can be perceived both before or after use of the tampon. For example, the distinctiveness may be conferred by treatment which stains the tail red after use but maintains the string in pristine condition, though both could be white initially. Or, the tail or string may change color during use due to body heat or moisture, for example.

The term “vaginal cavity,” “within the vagina,” and “vaginal interior,” as used herein, are intended to be synonymous and refer to the internal genitalia of the mammalian female in the pudendal region of the body. The term “vaginal cavity” as used herein is intended to refer to the space located between the introitus of the vagina (sometimes referred to as the sphincter of the vagina or hymeneal ring,) and the cervix. The terms “vaginal cavity,” “within the vagina” and “vaginal interior,” do not include the interlabial space, the floor of vestibule or the externally visible genitalia.

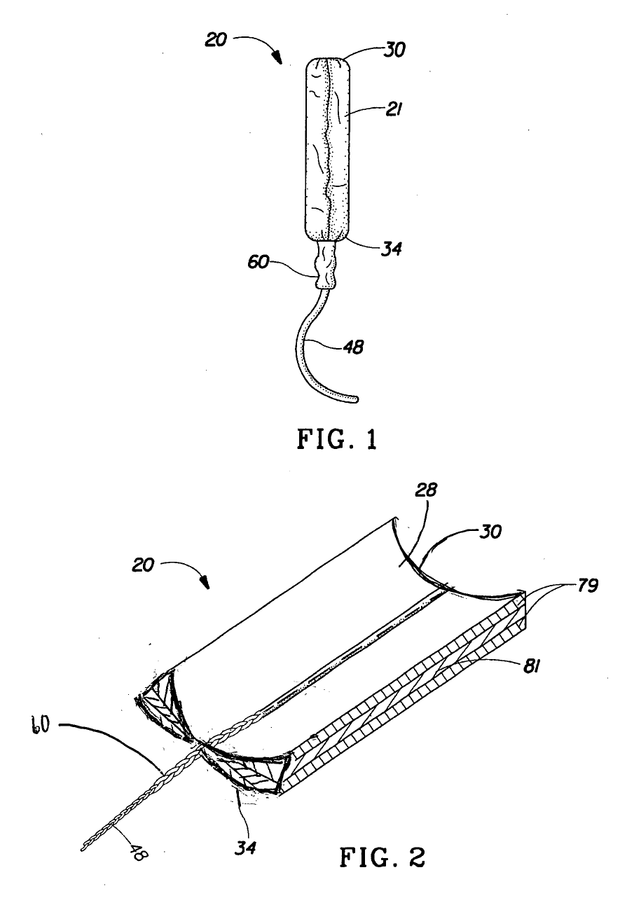

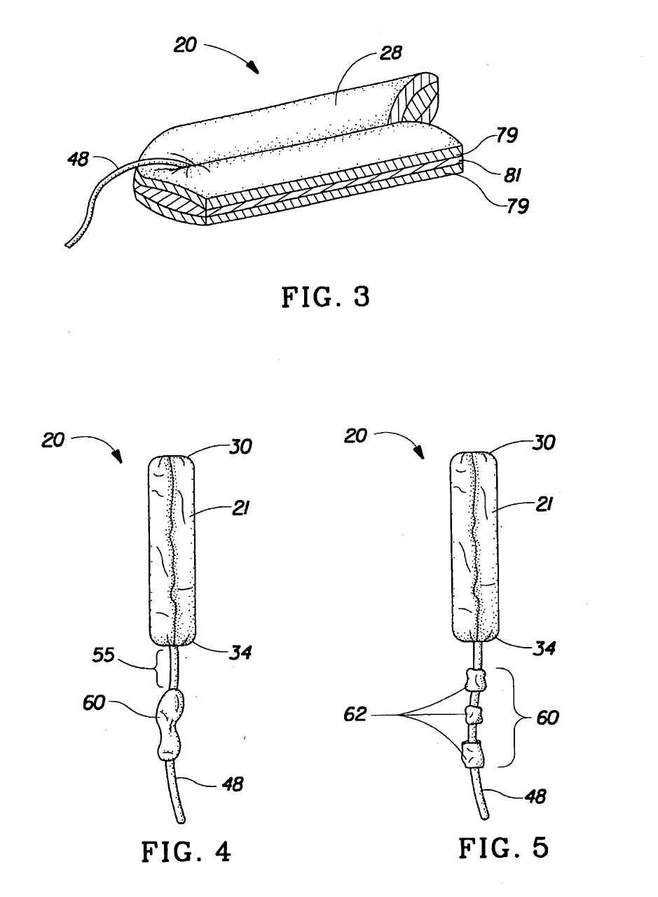

FIG. 1 shows on embodiment of the catamenial tampon 20 of the present invention. The present invention, however, is not limited to the structure having the particular configuration shown in the Figures. The tampon 20 of the present invention comprises a primary absorbent member 21, a secondary absorbent member 60, and a visually distinct withdrawal member 48. The visually distinct withdrawal member 48 is distinct from the secondary absorbent member 60 before and after use. The tampon 20 of the present invention can be any shape in the art and/or any type of tampon known in the art.

The primary absorbent member 21 of the tampon 20 shown in FIG. 1 has an insertion end 30 and a withdrawal end 34. The primary absorbent member 21 may be compressed into a generally cylindrical configuration in the radial direction, the axial direction, or in both the radial and axial directions. While the primary absorbent member 21 may be compressed into a substantially cylindrical configuration, other shapes are also possible. These may include shapes having a cross section which may be described as rectangular, triangular, trapezoidal, semi-circular, or other suitable shapes.

The primary absorbent member 21 of the tampon 20 of the present invention may be formed from any suitable tampon pledget, such as tampon pledget 28 shown in FIG. 2. The secondary absorbent member 60, described in greater detail below, is joined to either the visually distinct withdrawal member 48, the primary absorbent member 21 itself, or both. This joining of the secondary absorbent member 60 may occur subsequently to compression of the pledget 28 (FIG. 2) to form the primary absorbent member 21. In some variations it may be desirable to attach some or all of the secondary absorbent member 60 to the pledget 28, the visually distinct withdrawal member 48, prior to compression of the pledget 28 to a self-sustaining form into the primary absorbent member 21. In one method of making a tampon 20 of the present invention, described more fully below, the secondary absorbent member 60 is integral with the primary absorbent member 21 prior to compression of the pledget. In any of the above mentioned manners of construction, the secondary absorbent member 60 may not be compressed; or, if compressed, it is not compressed to the same degree as the primary absorbent member 21.

The tampon pledget 28 portion of the tampon 20 which will be compressed to form the primary absorbent member 21 may be any suitable shape, size, material, or construction. In the embodiment shown in FIG. 2, pledget 28 is a batt of absorbent material. While the pledget 28 shown in FIG. 2 is generally rectangular, other shapes such as trapezoidal, triangular, hemispherical, chevron shaped and hourglass shapes are also acceptable. The pledget 28 may be a laminar structure comprised of integral or discrete layers. The pledget 28, such as in FIG. 2, may comprise outer layers 79 and at least one intermediate layer 81 positioned between the outer layers 79. In other embodiments, the pad need not have a layered structure at all. The pledget 28 may comprise a folded structure, may be rolled, may comprise a “petal” structure or any other of the structures which are known in the art with respect to tampon pledgets 28. A more detailed description of liquid-absorbing materials and pledget shapes and dimensions can be found in currently pending and commonly assigned, U.S. patent Ser. No. 10/039,979, filed Oct. 24, 2001, entitled “Improved Protection and Comfort Tampon”, to Agyapong et al., Docket Number 8758.

A typical size for tampon pledget 28 prior to compression may be from about 40 mm to about 100 mm in length and from about 40 mm to about 80 mm in width. In general, the pledget material may be from about 40 mm to about 60 mm in length and from about 50 mm to about 70 mm in width. The typical range for the overall basis weight is from about 150 gsm to about 1500 gsm.

Tampon pledgets 34 may be constructed from a wide variety of liquid-absorbing materials commonly used in absorbent articles. Such materials include but are not limited to rayon (such as GALAXY Rayon SARILLE L rayon both available from Kelheim Fibers of Kelheim, Germany), cotton, folded tissues, woven materials, nonwoven webs, synthetic and/or natural fibers or sheeting, comminuted wood pulp which is generally referred to as airfelt, or combinations of these materials. Other materials that may be incorporated into the tampon pledget 20 including peat moss, absorbent foams (such as those disclosed in U.S. Pat. No. 3,994,298 issued to DesMarais on Nov. 30, 1976 and U.S. Pat. No. 5,795,921 issued to Dyer, et. al,) capillary channel fibers (such as those disclosed in U.S. Pat. No. 5,356,405 issued to Thompson, et. al on Oct. 18, 1994), high capacity fibers (such as those disclosed in U.S. Pat. No. 4,044,766 issued Kaczmarzk et al. on Aug. 30, 1977), superabsorbent polymers or absorbent gelling materials (such as those disclosed in U.S. Pat. No. 5,830,543 issued to Miyake, et al on Nov. 3, 1998).

The tampon 20 of the present invention may optionally include an overwrap comprising material such as, rayon, cotton, bicomponent fibers, polyethylene, polypropylene, other suitable natural or synthetic fibers known in the art, and mixtures thereof. In some embodiments, the tampon has a nonwoven overwrap comprised of bicomponent fibers that have a polypropylene core surrounded by polyethylene manufactured by Vliesstoffwerke Christian Heinrich Sandler GmbH & Co.KG (Schwarzenbach/Saale, Germany) under the tradename Sawabond 4313. In other embodiments, the tampon may comprise a nonwoven overwrap of a hydroentangled blend of 50% rayon, 50% polyester manufactured by Greenbay Nonwovens, Greenbay, Wis. In other embodiments, the tampon may comprises a nonwoven overwrap of 100% polyethelyene man manufactured by Greenbay Nonwovens, Greenbay, Wis. The overwraps may be treated to be hydrophilic, hydrophobic, wicking or non-wicking.

The tampon 20 of the present invention comprises a secondary absorbent member 60 that comprises a first color and the withdrawal member 48 comprises a second color wherein the first color and second color are different. The secondary absorbent member 60 and the withdrawal member 48 can both comprise a first color and a second color. The first color and the second color within the secondary absorbent member 60 and the withdrawal member 48 can be different. For example, the withdrawal member 48 can comprise a blue color and a white color. In another non-limiting example, the withdrawal member 48 can comprise a blue color and a white color and the secondary absorbent member 60 can comprise a red color and a yellow color. In one non-limiting example, the tampon 20 of the present invention can comprise a secondary absorbent member 60 that comprises a first color and a second color and the withdrawal member 48 can comprise a third color and a fourth color wherein the first color and second color are different from the third color and the fourth color. The number of colors which are different for the secondary absorbent member 60 and the number of colors which are different for the withdrawal member 48 are unlimited.

A visually distinct withdrawal member 48, examples of which are shown in FIGS. 1-2, may be joined to the tampon 20 for removal of the tampon after use. The tampon 20 of the present invention may also be provided with one or more or multiple visually distinct withdrawal members 48. For example, two visually distinct withdrawal members 48 may be attached down the length of the pledget 28 and extend from the withdrawal end 34 thereof. The visually distinct withdrawal member 48 is joined to at least the primary absorbent member 21 and extends beyond at least the withdrawal end 34 thereof. The visually distinct withdrawal member 48 may be integral with, or an extension of another element of the tampon, such as an overwrap as described above. The secondary absorbent member 60 may be joined to the visually distinct withdrawal member 48.

Visually distinct withdrawal members 48 useful in the present invention may be made of any suitable material known in the prior art and include cotton and rayon. In addition, the withdrawal means can take on other forms or compositions such as a ribbon, loop, tab, tape, embroidery floss, filament, braid, rope, string, or the like. The visually distinct withdrawal member 48 may be integral with the absorbent material. The visually distinct withdrawal member 48 or regions of the withdrawal member 32 may be treated to be non-absorbent.

The visually distinct withdrawal member 48 may be non-absorbent along at least the location of such attachment. As used herein, the term “non-absorbent” refers to a structure that does not retain a significant portion of deposited fluid in its structure. The entire visually distinct withdrawal member 48 may be made non-absorbent, if desired. The materials comprising the visually distinct withdrawal member 48 may be inherently non-wettable or hydrophobic, or they may be treated to provide such properties. For example, a coating of wax may be applied to the visually distinct withdrawal member 48 to decrease or eliminate its absorbency. Other means for providing a material suitable for use as a visually distinct withdrawal member 48 which is non-absorbent and/or non-wicking are known in the art. For example, U.S. Pat. No. 5,458,589 issued to Comin-DuMong describes one such approach. The visually distinct withdrawal member 48 need not necessarily be non-wicking, even if a non-absorbent visually distinct withdrawal member 48 is desired. For example, it may be desirable to provide a visually distinct withdrawal member 48 in which at least a portion of the cord has a tendency to wick deposited fluid upwardly toward the withdrawal end 34 of the primary absorbent member 21 and into the structure thereof.

The visually distinct withdrawal member 48 may be joined to any suitable location on the tampon 20. In the embodiment shown in FIGS. 1-2, the visually distinct withdrawal member 48 is joined to the primary absorbent member 21 and extends freely beyond the withdrawal end 34 of the primary absorbent member 21. The visually distinct withdrawal member 48 can be attached to the tampon pledget 28 while the pledget 28 is still uncompressed as shown in FIG. 2. The visually distinct withdrawal member 48 may be attached along the entire length or one major surface of the pledget 28 (such as by stitching) and hang free from one end, such as the withdrawal end 34. The visually distinct withdrawal member 48 may be attached in any suitable manner known in the art including sewing, adhesive attachment, or a combination of known bonding methods including the method disclosed in currently pending, commonly assigned, U.S. patent application Ser. No. 10/610,075, filed Jun. 30, 2003, entitled “Method and Apparatus for Cord Attachment” to Sargent, et al., Docket No. 9319.

The visually distinct withdrawal member 48 may be sprayed, soaked, painted, dyed, screen printed, laser printed, coated, laminated, vapor deposited, or otherwise marked. Any dye, paint or pigment may be used. Suitable dyes, paint and pigments are permanent, so as to not to stain the user or garments of wearer, and nontoxic and nonirritating to the wearer. The visually distinct withdrawal member 48 may be treated with a variety of adjuvants to become visually distinct. Such adjuvants include but are not limited to dyes, organic dyes, fiber sensitive dyes, such as dichlorotriazine and bis(aminonicotinotriazine), fluorescent reagents, cyclic peroxides, peroxyoxalates, organo-phosphors, bleaches, whiteners, metallic pigments and the like.

The visually distinct withdrawal member 48 may be printed with a pattern to become visually distinct. The patterns may be include but not limited to flowers, ovals, circles, rectangles, trapezoids, triangles, cones, alphabet letters, Trademarks, animal prints, stripes, lines, and mixtures thereof.

The visually distinct withdrawal member 48 may be made with material which lends to its visual distinctness. Such materials include materials such as acrylic, canvas, cashmere, chiffon, COOLMAX, corduroy, cotton, denim, elastane, flannel, fleece, fur, gabardine, GORTEX, jacquard, lace, leather, linen, lycra, merino, microfiber, nylon, polyamide, plastics, polyester, poplin, PVC, rayon, satin, shearling, silk, silver, spandex, straw, suede, taffeta, twill, velvet, viscose, wool and the like. Moreover, the material can be any transparent material such as nylon (fishing line), polyethylene, and Dacron. The material can be a monofilament, cofilament, fused, and/or braided.

The material that comprises the visually distinct withdrawal member 48 may subjected to mechanical means which lends to cause the visually distinctiveness. Such mechanical process include but are not limited to braiding, crimping, curling, MICREXing, ring rolling process (as described in U.S. Pat. No. 4,107,364 issued to Sisson on Aug. 15, 1978, U.S. Pat. No. 4,834,741 issued to Sabee on May 30, 1989, U.S. Pat. No. 5,143,679 issued to Gerald M. Weber, et al. on Sep. 1, 1992, U.S. Pat. No. 5,156,793 issued to Kenneth B. Buell, et al. on Oct. 20, 1992, and U.S. Pat. No. 5,167,897 issued to Gerald M. Weber, et al. on Dec. 1, 1992) and the “SELFing” process (as described in U.S. Pat. No. 5,518,801 issued to Chappell, et al. on May 21, 1996.)

The tampon 20 of the present invention is also provided with a secondary absorbent member 60. The secondary absorbent member 60 may comprise one piece of material as shown, for example, in FIG. 1, or may comprise multiple discrete pieces as shown, for example in FIG. 5. The size of the secondary absorbent member 60 may vary according to its shape. For example, in the embodiment shown in FIG. 1 the secondary absorbent member 60 is generally cylindrical and elongated. The length of the secondary absorbent member is measured in the direction generally parallel to a line running through the axis of the tampon extending through the insertion end and withdrawal end of the primary absorbent member 21.

The secondary absorbent member 60 of the present invention comprising material such as, rayon, cotton, bicomponent fibers, polyethylene, polypropylene, other suitable natural or synthetic fibers known in the art, and mixtures thereof. The secondary absorbent member 60 may be attached or integral with the primary absorbent member 21. The secondary absorbent member 60 may be arranged in a wide variety of shapes and configurations and may be generally cylindrical, spherical, semi-spherical, disc-like, planar, rectangular, “sheet-like,” “skirt-like” in shape, or comprise “tufts” or “whips” of material. The secondary absorbent member 60 may range from 10 mm to 40 mm in length. In some embodiments, the secondary absorbent member 60 may be 20 mm to 25 mm in length. The secondary absorbent member 60 may range from 6 mm to 20 mm in width. In some embodiments, the width may range from 5 mm to 16 mm. The secondary absorbent member 60 may range in thickness from 0.5 mm to 10 mm. In some embodiments, the secondary absorbent member 60 may range in thickness from 1 mm to 3 mm. The secondary absorbent member 60 may be single ply or multiple plys.

The secondary absorbent member 60 may be absorbent and/or hydrophilic. In some embodiments, the secondary absorbent member 60 may have an advancing contact angle greater than the advancing contact angle of the primary absorbent member 21 and/or the visually distinct withdrawal member 48, such that fluid is preferentially directed toward and absorbed by the primary absorbent member 21. Optionally, the secondary absorbent member 60 may be treated to make it less absorbent than the primary absorbent member 21.

The secondary absorbent member 60 is joined to the tampon 20 at any suitable location generally proximate the withdrawal end 34 of the primary absorbent member 21. In the embodiment shown in FIG. 1, the secondary absorbent member 60 is joined to the visually distinct withdrawal member 48 and is generally centered axially around the withdrawal member 48. It is also possible to attach such a mass of secondary absorbent member 60 to the withdrawal end 34 of the primary absorbent member 21 either in addition to, or in lieu of, attachment to the visually distinct withdrawal member 48.

The secondary absorbent member 60 may be attached to the visually distinct withdrawal member 48 (or other withdrawal mechanism) or the withdrawal end 34 of the primary absorbent member 21, or both, may be attached in any suitable manner known in the art including sewing, adhesive attachment, or a combination of known bonding methods including the method disclosed in currently pending, commonly assigned, U.S. patent application Ser. No. 10/610,075, filed Jun. 30, 2003, entitled “Method and Apparatus for Cord Attachment” to Sargent, et al., Docket No. 9319.

The secondary absorbent member 60 may also be integral with any other component of the tampon 20. For example, the secondary absorbent member 60 may comprise an extension of the primary absorbent member 21. The secondary absorbent member 60 may be in the form of a sheet or layer of absorbent material. For example, one or more internal layers 81 such as that shown in FIG. 2 could extend beyond the withdrawal end 34 of the pledget 28 either along its entire width or a portion of its width to form the secondary absorbent member. The secondary absorbent member could be comprised of an extension of the overwrap.

The secondary absorbent member 60 may be absorbent. The specific absorbency of the secondary absorbency material 60 may be less than, equal to, or greater than that of the primary absorbent member 21. In FIG. 5, the secondary absorbent member 60 is comprised of multiple pieces of absorbent material; the primary absorbent member 21 has a total absorbency which exceeds the total combined absorbency of such multiple pieces of secondary absorbent member 60.

In FIG. 4, the secondary absorbent member 60 is joined to the visually distinct withdrawal member 48. The secondary absorbent member 60 in FIG. 4, however, is positioned at a location slightly lower along the visually distinct withdrawal member 48 than is the case with FIG. 1, thereby creating a “gap” 55 between the withdrawal end 34 of the primary absorbent member 21 and the uppermost part of the secondary absorbent member 60. The length of the gap 55 along the visually distinct withdrawal member 48 may be in the range of from about 1 mm to about 15 mm. As shown in FIG. 5, the secondary absorbent member 60 may comprise a series of discrete pieces of absorbent material 62. In the embodiment shown in FIG. 5, these discrete pieces of absorbent material 62 are attached along the visually distinct withdrawal member 48 of the tampon 20.

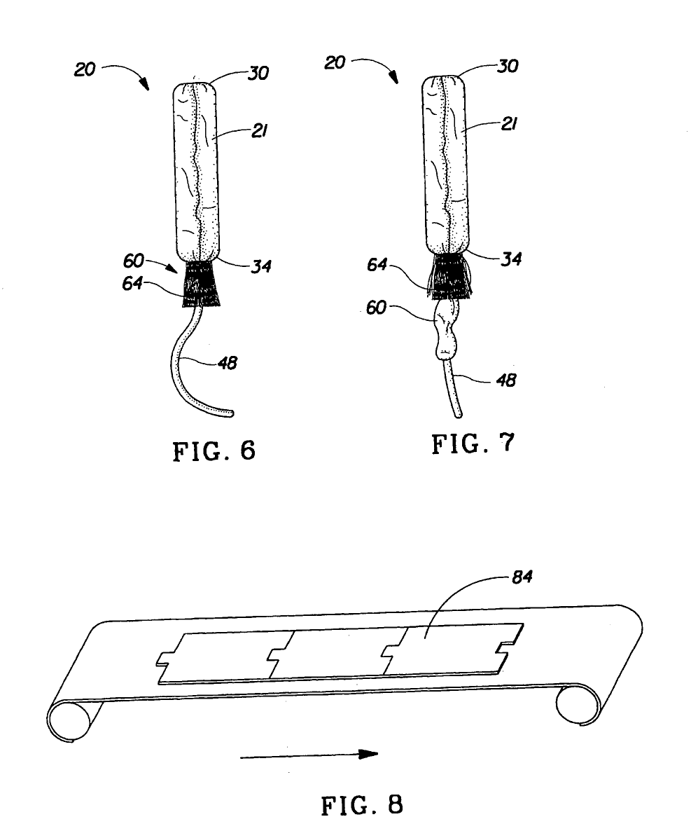

In FIG. 6, the secondary absorbent member 60 is joined to the withdrawal end 34 of the primary absorbent member 21. The secondary absorbent member 60 shown in FIG. 6 is in the form of a series of absorbent strands or “tufts” 64 of material depending from the withdrawal end 34 of the primary absorbent member 21. These absorbent strands or tufts 64 can also be substantially continuous or be constructed from one piece of material arranged in the form of a “skirt” around the withdrawal end 34 of the primary absorbent member 34.

In FIG. 7, it is possible to combine different variations on the form of the secondary absorbent member 60 described above in the same tampon 20. For example, FIG. 7 shows the combination of a single piece of secondary absorbent member 60 joined to the visually distinct withdrawal member 48 used in combination with additional secondary absorbent member 60 in the form of absorbent strands or tufts 64.

In any of the embodiments described, the visually distinct withdrawal member 48 of the tampon 20 extends below the withdrawal end 34 of the primary absorbent member 21 by about 10 cm.

Both the primary absorbent member 21 and secondary absorbent member 60 may reside entirely within the vaginal cavity of the wearer during use of the tampon 20. This is achieved by the relative closeness of the secondary absorbent member 60 to the withdrawal end 34 of the primary absorbent member 21, as well as the relative size compared to the overall size of the tampon 20. Generally, only the visually distinct withdrawal member 48 resides externally to the orifice of the vagina.

The fluid absorbed and retained by the tampon 20 will ultimately be retained in the primary absorbent member 21. The secondary absorbent member 60 may be more hydrophilic than the visually distinct withdrawal member 48. The visually distinct withdrawal member 48 may be made substantially hydrophobic. If the entire visually distinct withdrawal member 48 not less hydrophilic than the secondary absorbent member 60, at least portions of the visually distinct withdrawal member 48 are less hydrophilic than the secondary absorbent member 60.

For a more detailed description of hydrophilicity and contact angles see the following publications which are incorporated by reference herein: The American Chemical Society Publication entitled “Contact Angle, Wettability, and Adhesion,” edited by Robert F. Gould, and copyrighted in 1964; and TRI/Princeton Publications, Publication Number 459, entitled “A Microtechnique for Determining Surface Tension,” published in April 1992, and Publication Number 468 entitled, “Determining Contact Angles Within Porous Networks,” published in January, 1993, both edited by Dr. H. G. Heilweil.

The secondary absorbent member 60 may optionally be provided with a mechanism to preferentially direct acquired fluid toward the body of the primary absorbent member 21. Examples of such a driving force are the use of a hydrophilicity gradient as described above. Other mechanisms include a density or capilarity gradient, or an osmotic driving force. Capillary channel fibers may optionally be incorporated into the secondary absorbent member 60 in order to provide the driving force for acquired fluid described herein.

While several methods of making the tampon of the present invention should be apparent to one of skill in the art in light of the disclosure herein, following is a description of one method of making a tampon of the present invention.

To form a tampon ready for use, the tampon pledget 28 can be compressed and heat conditioned in any suitable conventional manner. Pressures and temperatures suitable for this purpose are well known in the art. The pledget 28 can be compressed in both the radial and axial direction using any means well known in the art. While a variety of techniques are known and acceptable for these purposes, a modified tampon compressor machine available from Hauni Machines, Richmond, Va., is suitable. If the secondary absorbent member 60 is attached to the tampon 20 after compression of the pledget 28, then no modification of the method of making a conventional compressed absorbent tampon is necessary (except of course that the secondary material is attached after the otherwise completed tampon is formed).

While several methods of making a tampon 20 of the present invention would be apparent to one of skill in the art in light of the disclosure herein, following is a description of one method of making a tampon of the present invention is a continuous operation in which the secondary absorbent member is integral with the tampon pledget 28.

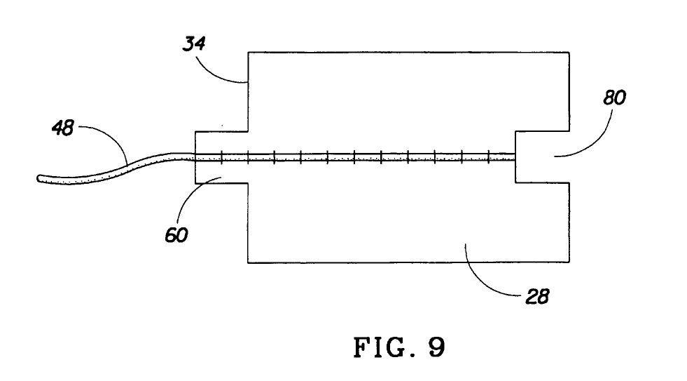

In such a method of making, a generally rectangular (or other suitably shaped) pad of absorbent material is formed. FIG. 8 shows a strip of absorbent material formed by a suitable carding process. The carding process results in a continuous strip of absorbent material 84 which may then be cut into individual absorbent pads, such as those shown in FIG. 9. The term “continuous strip” is intended to mean an elongated mass of absorbent material of sufficient length in the machine direction to be cut into two or more individual pledgets 28. The cutting operation may be performed (i.e. through the use of a suitably shaped cutting die) such that a “notch” 80 is formed in the insertion end of each pledget 28. This notch 80 will result in an integral piece of absorbent material which depends from the withdrawal end 34 of the pledget 28. The notch 80 may be generally square or rectangular as shown in FIG. 9 or may be triangular, semi-circular, trapezoidal, or any of the other shapes disclosed herein for the secondary absorbent member 60. This notch 80, as shown in FIG. 9 will serve as the secondary absorbent member 60 of the finished tampon. The individual pads are then provided with a suitable visually distinct withdrawal member 48 which may extend down the entire length of the pledget 28 and mass of secondary absorbent member 60.

During formation of the tampon 20, the pledget 28 is compressed as described above. Because the secondary absorbent member is smaller in the width dimension than the pledget 28, the radial compression of the pledget 28 will not substantially compress the secondary absorbent member 60. During the axial compression stage, if any, only the pledget 28 portion is compressed through the use of a suitable push-rod and the secondary absorbent member remains essentially non-compressed.

The tampon 20 of the present invention may be inserted digitally or through the use of an applicator. If the tampon 20 is to be used for digital insertion, it may be desirable to form the pledget from a layer of absorbent material which has been rolled into a cylindrical shape. When the tampons are intended to be digitally inserted, it may be desirable to provide a finger indent at the withdrawal end 34 of the tampon 20 to aid in insertion. A finger indent can be made using a compression rod. An example of finger indents is found in U.S. Pat. No. 6,283,952, filed May 5, 1997, entitled “Shaped Tampon,” issued to Child, et al.

Alternatively, the insertion may be aided through the use of any applicator adapted from the prior art for use a tampon 20. Prior art applicators of a “tube and plunger” type arrangement can be made of plastic, paper, or other suitable material. Additionally, a “compact” type applicator is also suitable. In some embodiments, where the tampon 20 of the present invention is shaped and provides aesthetic appeal to consumers, it is may be desirable to combine the shaped tampon with an applicator type which enables the user to observe at least a portion or the whole shape of the shaped tampon. Two techniques which allow the user to better notice the shape of the tampon are to either make visual observation possible through the use of a translucent or even transparent applicator materials, or to provide a tampon applicator insertion end that better follows and hence better displays the profiled shape of the enclosed shaped tampon than the typical commercial tampon applicators comprising straight-walled cylindrical inserter tubes often made from molded plastic or laminated cardboard tubes. These techniques may be found in currently pending and commonly assigned, U.S. patent application Ser. No. 10/150,055, filed Mar. 18, 2002, entitled “Shaped Tampon,” to Kollowitz, Docket No. 8888.

The tampons 20 of the present invention can optionally employ wrappers which are tightly conforming to the outer surface of the tampon 20 in order to visually show the consumer the tampons packaged therein. Tightly conforming wrappers are particularly useful when the shaped tampons are intended to be used digitally and therefore are not housed in an applicator prior to use. The wrappers should substantially enclose each individual tampon and are intended to be removed prior to insertion and use. “Tightly conforming” means that there is substantially no visually noticeable void space between the wrapper and the tampon 20. In some embodiments of the present invention some regions of the wrapper material may provide additional functional benefits, such as cord deployment means. Since tampons 20 can be made by compressing fibrous absorbent material into a self-sustaining shape, the tightly conforming wrapper can optionally be used to act with a certain compressing force on the outer surface of the tampon, which will aid maintaining said self-sustaining shape and by counteracting the expansion of the compressed material which otherwise. Such wrappers are discussed in detail currently pending and commonly assigned, U.S. patent application Ser. No. 10/150,055, filed Mar. 18, 2002, entitled “Shaped Tampon,” to Kollowitz, Docket No. 8888.

All documents cited in the Detailed Description of the Invention are, in relevant part, incorporated herein by reference; the citation of any document is not to be construed as an admission that it is prior art with respect to the present invention. To the extent that any meaning or definition of a term in this written document conflicts with any meaning or definition of the term in a document incorporated by reference, the meaning or definition assigned to the term in this written document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. A tampon comprising:
a primary absorbent member constructed from an absorbent material compressed to a self-sustaining form, said primary absorbent member having an insertion end, and a withdrawal end;
a secondary absorbent member attached to said primary absorbent member proximate to said withdrawal end of said primary absorbent member; and
a withdrawal member being visually distinct from the secondary absorbent member;
wherein the visual distinctiveness is due to a difference in color, shade, pattern, reflectance, opacity, translucence, opalescence, fluorescence, luminescence, phosphorescence, chemiluminescence, whiteness and the mixtures thereof.

2. The tampon of claim 1 wherein said withdrawal member is transparent.

3. The tampon of claim 1 wherein said withdrawal member has a first color and a second color wherein said first color and said second color are different.

4. The tampon of claim 3 wherein said first color and said second color are arranged in a pattern.

5. The tampon of claim 4 wherein said pattern is selected from the group consisting of stripes, spiral lines, diagonal lines, straight lines, checkerboard and mixtures thereof.

6. The tampon of claim 1 wherein said withdrawal member is attached to said primary absorbent member proximate to said withdrawal end.

7. The tampon of claim 1 wherein said withdrawal member is attached to said secondary absorbent member proximate to said withdrawal end.

8. A tampon comprising:
a primary absorbent member constructed from an absorbent material compressed to a self-sustaining form, said primary absorbent member having an insertion end, and a withdrawal end;
a secondary absorbent member fixedly attached to said primary absorbent member proximate to said withdrawal end of said primary absorbent member;
said secondary absorbent member comprised of a first color; and
a withdrawal member comprised of a second color;
wherein said first color is different from said second color.

9. The tampon of claim 8 wherein said withdrawal member is attached to said primary absorbent member proximate to said withdrawal end.

10. The tampon of claim 8 wherein said withdrawal member is attached to said secondary absorbent member proximate to said withdrawal end.
